(19) United States
(12) Patent Application Publication
(10) Pub. No.: US 2010/0261223 A1
(43) Pub. Date: Oct. 14, 2010

(54) DEVICE FOR DETECTING COMPONENTS IN A FLUID

(76) Inventors: Stefan Margraf, Frankfurt,/Main (DE); Martin Scholz, Oberursel (DE)

Correspondence Address: Muncy, Geissler, Olds & Lowe, PLLC, 4000 Legato Road, Suite 310, FAIRFAX, VA, 22033, US

(21) Appl. No.: 12/740,817

(22) PCT Filed: Oct. 31, 2008
(86) PCT No.: PCT/EP08/09219

§ 371 Date: Apr. 30, 2010

(30) Foreign Application Priority Data
Oct. 31, 2007 (EP) 07021342.6

(51) Int. Cl. C12Q 1/02; C12M 1/34

(52) U.S. Cl. 435/29; 435/287.2; 435/288.7; 435/287.1; 435/288.1

(57) ABSTRACT

A device and a method for detecting components in blood, in particular for determining the concentration of components in blood or water is provided. Moreover, a use of a device and/or a method for determining components in blood is provided and a kit including the device and a fluorescence standard.

This nonprovisional application is a National Stage of International Application No. PCT/EP2008/009219, which was filed on Oct. 31, 2008, and which claims priority to European Patent Application No. EP 07021342.6, which was filed on Oct. 31, 2007, and which are both herein incorporated by reference.

### BACKGROUND OF THE INVENTION

1. Field of the Invention

The present invention relates to a device and a method for detecting components in blood or water, in particular for determining the concentration of components in blood or water. Moreover, the present invention relates to the use of a device or a method for determining components in blood. The invention finally relates to a kit comprising said device and a DNA standard.

2. Description of the Background Art

In the following, various documents including patent applications and instruction manuals are cited. Although not being considered relevant to the patentability of the present invention, the entire content of these documents is incorporated by reference. In particular, all cited documents are incorporated by reference to the same extent as if each individual document were designated as being specifically and individually incorporated by reference.

Methods for medical diagnosis are used as a matter of routine for being able to estimate the clinical status of patients based on the determination of the presence and/or concentration of substances dissolved in the blood. So far, analyses of the blood plasma and/or serum have had to be carried out in a centrifugal step for being able to separate the blood plasma to be analyzed from the solid components of the blood. Apparently, no suitable alternative methods or a device for separating the solid blood components exist. Centrifugation is not only time-consuming, but in particular also in developing regions it is a great obstacle because in these regions often not only suitable centrifuges are lacking but also the energy for operating them. Moreover, in many regions of the world there is often no specialized staff that can readily carry out blood analyses by using known means.

Therefore, there is a need for a device and a method allowing the analysis of substances contained in blood plasma and/or serum, in particular without centrifugation. Moreover, a corresponding device or method should also allow untrained staff to carry out such analyses quickly, cleanly and correctly without having to be trained for a long time.

U.S. Pat. No. 5,186,843 discloses a material for separating plasma or serum from blood. The material comprises glass microfibers, cellulose fibers and synthetic textile fibers. This medium is used for obtaining small amounts of plasma collecting within the layer used for separation. However, the still necessary collection and further handling of the liquid are an obstacle to a quick and clean diagnosis.

U.S. Pat. No. 4,816,224 describes a device for separating plasma or serum from whole blood, which can be designed in different manners. Glass fibers having a large volume are used for separation. The device can comprise a plurality of filter layers.

US 2006/0029923 describes a device which allows plasma or serum to be separated from solid blood components by means of a filter membrane in the device and pressure. The corresponding device can comprise a detection strip for detecting components in the blood by immunochemical detection methods. However, by means of these strips no direct detection is possible without the staff having to carry out further steps or without the use of further auxiliary means and/or substances. The device is primarily intended for obtaining blood plasma or serum for further tests.

Thus, devices of the above-mentioned kind are mainly used for providing small amounts of plasma or serum for further analyses.

By means of present methods and devices for testing plasma or serum and/or other body fluids, which are intended for obtaining a measuring result quickly, often only index values can be obtained, and frequently only statements as to the presence or absence of components of a fluid to be measured can be made. For example, test strips only indicate whether a substance is present within the detection limit or not. However, these methods and devices are often not suitable for the diagnosis of specific diseases and/or conditions in samples from patients, because only precise measuring values as to the concentration or content of a substance in a fluid, e.g. blood, provide information about the specific condition of the patient and suitable treatment methods.

### SUMMARY OF THE INVENTION

Therefore, it is an object of the invention to provide a device and a method which overcome the disadvantages of the prior art. In particular, it is an object to provide a device by means of which plasma or serum can be separated from whole blood and substances contained in the serum or plasma can be analyzed without centrifugal steps or similar laboratory processing steps of the obtained serum or plasma being necessary. Further or additional objects of the present invention are the provision of a device that can be handled easily, safely and reliably and a method that can be carried out easily and can be used or carried out in particular by laymen or staff that is not medically trained, the provision of a device that can be produced and/or stored easily and in a cost-efficient manner, as well as the provision of a corresponding kit.

This object is achieved by the features defined in the claims.

The present invention preferably relates to a device for detecting components in blood, in particular for determining the concentration of components in blood, the device comprising a measuring region, a filter and/or filter region, at least one detection reagent for interaction with the components, an opening for introducing a fluid, the filter being arranged between the opening and the measuring region, as well as a fluid inlet region, wherein the fluid inlet region and/or the filter region are formed or limited at least partly by a preferably elastic region, which preferably comprises a film. The fluid inlet region is arranged preferably between opening and filter.

When measuring autofluorescence or the absorption of components of fluids, a detection reagent can also be omitted.

“Detection” of, e.g., substances means in particular the determination of the presence or absence of a substance. Here, the detection limit of the measuring method determines the result within the desired accuracy.

The “concentration” of substances is independent of the volume, in particular as compared to the absolute amount of substance.

In the present invention, “components in blood” are in particular substances that are present and/or dissolved in blood. The substances can be in particular organic or inorganic or a mixture thereof. Basically, also components of other fluids can be determined by means of the device of the present invention. Here, fluids are understood as liquids comprising solid components or suspensions. Preferably, the fluid is a body fluid. Body fluids are, e.g., blood, liquor, urine, serous liquids, saliva, sperm or pathological stool. Preferably, the fluid is water, in particular from taps, streams or lakes etc. Here, it can be determined, for example, whether a measured DNA correlates with the contamination of the water by microorganisms.

A “measuring region” in the meaning of the present invention is in particular a space, preferably having a defined or definable volume, in which at least one component of a fluid is determined. It is preferably at least partially transparent and suitable in particular for determining the substance or the component preferably by means of optical methods. The measuring region is preferably located downstream of the filter and is in fluid communication therewith. It can directly adjoin the filter or it can be spaced therefrom.

The region which directly adjoins the filter and receives the filtrate is also called filtrate region. The filtrate region can correspond either fully or partly to either the measuring region or to the element(s), which is/are arranged downstream of the filter, receive(s) the filtrate and is/are arranged upstream of the measuring region.

Here, the terms “upstream of” and “downstream of” and the like are used in view of the fluid flow direction from the opening through the filter into the measuring region.

For use with preferred detection reagents which are, e.g., excited by light of specific wavelength ranges and emit light of specific wavelength ranges, the measuring region preferably allows the transmission of, e.g., visible light as well as the light of the emission wavelengths.

The filter of the present invention can comprise or consist of any material suitable for separating blood or other fluids comprising solid components. For example, the filter can comprise or be based on polyethylene terephthalate (distributed, e.g., by the company Sekisui in a product for obtaining serum) or polysulphone (e.g., Vivid Plasma Separation Membrane (previously BTSSP) distributed by the company Pall).

Known suitable filters and/of filter materials are described, e.g., in U.S. Pat. No. 4,816,224, U.S. Pat. No. 5,186,843 or US 2006/0029923.

Preferably, the filter comprises glass fibers but is not made completely of glass fibers. More preferably, the volume or weight proportion of glass fibers ranges between 0% and 90%, 80%, 70%, 60%, 50%, 40%, 30%, 20% or 10% or it ranges between about 5% and 50% of the filter, preferably between about 10% and 50%. Most preferable is a filter material without glass fibers. For filtering blood, the filter material should not cause hemolysis and not bond analytes.

In the present invention, “detection reagent” is in particular a substance for detecting the presence and/or concentration of another substance, preferably a substance in a fluid, e.g. blood, plasma or water. A detection reagent is preferably capable of allowing or causing a detectable reaction under specific conditions. A detection reagent preferably interacts directly with the substance to be determined. It either forms a covalent or non-covalent bond with this substance. Preferably, fluorescence of the detection reagent is increased only after bonding to the analyte.

The device comprises an opening for introducing a fluid, wherein the filter is arranged between the opening and the measuring region. Moreover, the device comprises a fluid inlet region.

The fluid inlet region is preferably arranged between the opening and the measuring region, more preferably between the opening and the filter.

In the following, the region directly upstream of and/or directly downstream of the filter is also referred to as filter region. More preferably, the filter region further comprises the filter, parts or the entirety of the fluid inlet region, the filtrate region and/or the measuring region.

The filter region preferably has a volumetric capacity of between about 200 and about 2000 μl. Depending on the amount and nature of the fluid or preferably the blood, up to 200 μl of plasma or serum can be gained for the measurement in the measuring region. Preferably, the device of the invention allows the provision of a volume to be measured in the measuring range between about 15 μl and about 80 μl, preferably between about 20 μl and about 60 μl, and most preferably of about 40 μl of serum or plasma.

The fluid inlet region and/or filter region is formed or limited at least partially or completely by a, preferably elastic, film. The film is preferably made of an artificial or natural polymer or also copolymer. Examples of such polymers and copolymers are PVC, ethylene vinyl acetate copolymer film, polyethylene, polyethylene copolymer, polypropylene, polypropylene copolymer, a mixture of these polymers or also block polymers, coextrusions, multi-layer films as well as also films having no smooth surface. The film preferably limits the filter region. In the filter region the filter is preferably connected such with the film adjoining the filter region, e.g. welded or glued, that the region downstream of the filter, which receives the filtrate, is separated completely from the fluid inlet region by the filter. Before filling the device of the invention, the filter can contact the film limiting the filter region in a space-saving manner, e.g., in folded or also flat form. Thus, before filling, the fluid inlet region and the region downstream of the filter has only a small and preferably no inner volume. When the fluid inlet region is filled with the sample, the pressure with which the sample is introduced and the elasticity of the film cause the fluid inlet region surrounded by the film to expand. This pressure is held or stored such in the fluid inlet region that the sample is pressed through the filter. By filtration, the region downstream of the filter is filled with filtrate. If this region is also surrounded by the elastic film, this preferred embodiment of the invention leads to a particular small dead space volume on the side of the filtrate and thus allows in a particularly advantageous manner the use of very small amounts of filtrate. Moreover, the fragile filter membrane clings preferably to the inner surface of the film limiting the little bag and thus is advantageously supported mechanically. The filling and the elastic behavior of the film as well as the preferred presence of an inlet valve lead to a permanent and pushing pressure difference over the filter membrane, which then causes filtration even if the syringe used for filling is removed again.

The device of the invention is preferably a ready-to-use device for easily and reliably detecting, and in particular determining the concentration of, the components without comprehensive preparative measures. The present invention is in particular advantageous in that a device is provided which is small, can be read by means of commercially available devices and/or combines the separation of the solid components of fluids, preferably of blood, with the simultaneous measurement of components contained in the fluid phase. Thus, not only the centrifugation step which had so far been necessary for separating, e.g., solid blood components from serum or plasma is saved, but also untrained staff can analyze fluids in diagnostic processes because of the easy and safe handling. It is a further advantage that it is now possible to determine the concentration of components exactly, for example for estimating the condition of patients after operations or in case of specific medical conditions. Finally, the present device allows the immediate measurement of the components of interest (ready-to-use) without further treatment and/or delay or further necessary measuring steps.

In accordance with the present invention, the components are preferably determined in serum or plasma, or blood serum or blood plasma.

“Plasma” preferably means in particular the liquid phase of the blood which was separated from solid components such as cells (erythrocytes, white blood cells, etc) and can still coagulate.

“Serum” preferably means in particular the liquid part of the blood obtained after coagulation of the blood by separating the cellular components mixed with thrombocytes and coagulation factors to the coagulum.

In accordance with the present invention, basically any substance can be detected by means of suitable detection reagents. Preferably, the component to be detected or determined is a substance occurring in organisms. Substances occurring in organisms can be of organic or inorganic nature. For example, minerals or mineral salts, trace elements, inorganic ions, metals and heavy metals, etc, are inorganic.

Organic substances can belong to different substance classes. A group of substances is formed by proteins, which also include enzymes. Enzymes convert their substrate into a final product. In accordance with the present invention, preferably both the enzyme and the substrate and/or the final product can be determined. Also non-enzyme proteins can preferably be detected or determined by means of the device of the present invention. Further groups of organic substances comprise vitamins and coenzymes, nucleic acids, cytokines, hormones, histones, peptides, sugar, etc.

A further group of substances is formed by nucleic acids comprising DNA and RNA in its single- and/or double-stranded forms.

Preferably, the component to be detected or determined is a biologic molecule selected from the group comprising DNA, RNA, proteins, hormones, cytokines. The latter substances can be free or in association with other proteins. For example, DNA in plasma or serum can also occur as a complex with histones and/or elastase as well as micro satellites. Moreover, the component to be detected or determined is preferably the DNA/RNA of intact or non-intact bacteria, viruses and/or parasites of water samples contaminated with other particles. The particles are held back by the filtering process, while the nucleic acid contained in microorganisms can be measured by using suitable fluorescent dyes.

The component to be detected or determined is preferably a medicament or drug. A medicament or drug is a substance administrable to an individual for treating a medical condition or a disease. A medicament or drug can also be the above-mentioned biological molecules.

Most preferably, DNA is determined or measured. The filter used in accordance with the present invention preferably bonds only little or no DNA. The bound part is preferably less than 30%, preferably less than 20%, most preferably less than 10% of the DNA contained in the fluid or blood.

In view of quality and quantity, DNA can be detected in many ways. These ways include the PCR method as well as the detection by detectable agents specifically interacting with DNA. The present invention preferably allows the detection of non-cell-bound DNA. Non-cell-bound DNA can be detected by means of different methods. In addition to the measurement of the fluorescence after addition of an intercalating dye, also the measurement of the UV absorption of DNA is used for determining the concentration. The sample volumes necessary for this purpose have meanwhile reached a low μl range (e.g. measurement by NanoDrop in the range of 2 μl and higher). The lower measuring limit for DNA is presently at about 10 ng/ml for the fluorescence method.

After relatively severe operations, in particular also with heart-lung machine, but also after accidents with multiple traumas, sepsis, burns as well as after ischemia/reperfusion disease (after arterial obstruction), strong, often excessive activations of the immune system occur. Examples of medical states, which are, however, not exhaustive, are operation, multiple trauma, soft-tissue trauma, ischemia/reperfusion disease, infarct, ischemia, embolism, infection, sepsis, transplantation, intoxication, eclampsia, side effects of medicaments or transfusions. They can cause temporary or permanent damage to organs but also be lethal. The cellular component of this immunological response is arranged by neutrophilic granulocytes. For estimating the consequences of the activation it is important to be promptly informed about the extent of the situation.

As disclosed in U.S. 60/827,571, the concentration of non-cell-bound DNA released by granulocytes in the blood increases in case of specific pathologic events which are not caused by cancer or pathogens. Depending on the pathologic state, this non-cell-bound DNA can be present freely and/or in the form of so-called NETs (Neutrophil Extracellular Traps), DNA networks decorated with proteins, which in this case are not only produced for fighting microorganisms but are also generally released in the course of immunological responses caused by specific pathologic events. While parts of these NETs stick to the capillaries, a further part breaks off caused by the blood flow and can be found in the plasma and serum.

The present invention provides a device and a method for detecting the NETs which are released already within minutes after activation of the granulocytes producing them. The detection in samples of patients can lead to timely reactions of the attending doctors which can safe lives.

Further free DNA in the blood is released by dead cells. These cells can have died by apoptotic or necrotic events. In the course of some therapies, for example of cancer with chemotherapy or radiotherapy, the apoptosis of cells, i.a. blood cells, is induced. The success of the therapy thus can be detected directly and in a finely stepped manner by means of the present invention.

Preferably, the presence and/or concentration of the component in the measuring region can be determined, preferably depending on the detection reagent used, by means of luminescence, fluorescence, chemiluminescence, electrochemiluminescence, spectral absorption photometry, autofluorescence and/or bioluminescence.

Preferably, the filter is realized so as to separate solid components of the fluid flowing through the filter, and in particular separate the solid and liquid phases of the fluid from each other. Some filter materials can turn out to be very brittle or fragile, so that stabilization is necessary. Depending on the nature of the filter, it can comprise a stabilizing element. Stabilizing elements are preferably stabilizing edges or frames or applied or integrated networks of a stable material, e.g. metal or synthetic material.

Preferably, the fluid inlet region of the device, which is preferably comprised by the filter region, is realized such that a pressure above ambient pressure is exerted on the fluid. The device is preferably configured such that such a pressure is built up during introduction of the fluid, preferably by means of a syringe or the like.

The device is preferably realized such that the fluid is introduced under pressure through the filter into the measuring region. This pressure can preferably be applied manually, e.g. by the piston of a syringe containing the unfiltered fluid to be measured.

According to a preferred embodiment of the invention, a pressure lying below ambient pressure, preferably a vacuum, is prevailing in the device. When the sample to be determined is brought in contact with the device, the low pressure or vacuum causes the sample to be transported or sucked into the device without any pressure being applied from outside and the solid components to be separated from the fluid inside the device. The measuring region preferably consists of a preferably elastic material which aims at taking a defined position or shape. Preferably, the device is made of an elastic material which is deformed in such a manner that the inner volume of the measuring chamber is smaller than that of the defined position so that it unfolds, e.g., when the sample is introduced or sucks in the sample during unfolding. Moreover, the measuring region is preferably made of an inelastic material, wherein the region inside the measuring region has a pressure lower than ambient pressure before the sample enters the device.

In a further preferred embodiment, the detection reagent is provided in the measuring region or in a cavity/lumen arranged shortly upstream of the measuring region and extending preferably between filter and measuring region.

In a further preferred embodiment, the detection reagent is provided in the filter or close to the filter.

In a further preferred embodiment, the detection reagent interacts directly or indirectly with the component to be detected. A direct interaction preferably takes place if DNA is detected by means of reagents bonding to the DNA, wherein bonding can be covalent or non-covalent. Specific substances intercalate in double-stranded or also single-stranded DNA without covalently bonding to the DNA (intercalators). Partly, only this imparts the property of fluorescence to the substances. By accumulation in the DNA and irradiation with light of specific wavelengths, these substances emit light of different wavelengths, which can be measured quantitatively and correlates with the amount of DNA. Fluorescent dyes can also be different from intercalators and can be brought in contact with the DNA to be measured by chemical modification. In another preferred embodiment, the detection reagent is selected from the group which also comprises cyanine dyes (Pico-Green™ ([2-[N-bis-(3-dimethylaminopropyl)-amino]-4-[2,3-dihydro-3-methyl-(benzo-1,3-thiazol-2-yl)-methylidene]-1-phenyl-quinoline]; also cf. Zipper et al., 2004; Nucleic Acids Research 32(12)), TOTO®—(e.g. 1-1′-[1,3-propanediylbis[(dimethylimino)-3,1-propanediyl]]bis[4-[(3-methyl-2(3H)-benzothiazolylidene)methyl]]-tetraiodide), Alexa®—(e.g. 2,3,5,6-tetrafluorophenylester (Alexa Fluor® 488 5-TFP; a compilation of the Alexa dyes can be taken from Berlier et al. (2003); J Histo Cytochem 51(12):1699-712) or Sytox® dyes (to be purchased from Invitrogen) and SYBR® dyes (e.g. C₃₂H₃₇N₄S⁺ or [2-[N-(3-dimethylaminopropyl)-N-propylamino]-4-[2,3-dihydro-3-methyl-(benzo-1,3-thiazol-2-yl)-methylidene]-1-phenyl-quinoline]; to be purchased from Invitrogen; also cf. Zipper et al., 2004; Nucleic Acids Research 32(12)) or else ethidium bromide (e.g. C₂₁H₂₀BrN₃). A list of suitable cyanine dyes is disclosed in U.S. Pat. No. 5,436,134 and U.S. Pat. No. 5,658,751.

In accordance with the present invention, a substance is preferably detected quantitatively in the measuring region. If only one detection reagent is present, it is advantageous that when interacting with the target molecule, the detection reagent changes its properties so as to become detectable. As described above, intercalators cannot be detected before intercalating in nucleic acids, whereas in the unbound state they do not exhibit this property. The detection reagent used in accordance with the present invention can already be detectable before interaction with the target molecule, but it preferably changes its respective properties when interacting with the target molecule. This happens preferably by displacing the absorption maximum or the emission wavelength of a detection reagent.

The measurement of the non-cell-bound DNA preferably comprises the detection of the fluorescence emission after addition of a fluorescent dye to the plasma or serum.

Basically, all fluorophores can be used in accordance with the invention. The following list, which is not complete, shows examples of suitable fluorophores: 1,5 IAEDANS, 1,8-ANS, 4-methyl umbelliferone, 4′,6-diamidino-2-phenylindol, 5- (and 6-) carboxy-2′, 7′-dichlorofluorescein pH 9.0, 5-carboxy-2,7-dichlorofluorescein, 5-carboxyfluorescein (5-FAM), 5-carboxynaphthofluorescein (pH 10), 5-carboxytetramethylrhodamine (5-TAMRA), 5-FAM (5-carboxyfluorescein), 5-FAM pH 9.0, 5-HAT (hydroxyl tryptamine), 5-hydroxy tryptamine (HAT), 5-ROX (5-carboxy-X-rhodamine, triethylammonium salt), 5-ROX (carboxy-X-rhodamine), 5-ROX pH 7.0, 5-TAMRA (5-carboxytetramethylrhodamine), 6 JOE, 6-carboxyrhodamine 6G, 6-carboxyrhodamine 6G pH 7.0, 6-carboxyrhodamine 6G hydrochloride, 6-CR 6G, 6-HEX, SE pH 9.0, 6-JOE, 6-TET, SE pH 9.0, 7-AAD (7-amino-actinomycin D), 7-amino-4-methylcoumarin, 7-aminoactinomycin D (7-AAD), 7-hydroxy-4-methylcoumarin, 9-amino-6-chloro-2-methoxyacridine, ABQ, acid fuchsin, ACMA, ACMA (9-amino-6-chloro-2-methoxyacridine), acridine homodimer, acridine orange, acridine orange+DNA, acridine orange, DNA & RNA, acridine red, acridine yellow, acriflavin, acriflavin feulgen SITSA, aequorin, AFPs, Alexa 405, Alexa 430, Alexa 488, Alexa 546, Alexa 568, Alexa 594, Alexa Fluor 350™, Alexa Fluor 430 antibody conjugate pH 7.2, Alexa Fluor 430™, Alexa Fluor 488 antibody conjugate pH 8.0, Alexa Fluor 488 hydrazide water, Alexa Fluor 488™, Alexa Fluor 532™, Alexa Fluor 546™, Alexa Fluor 568 antibody conjugate pH 7.2, Alexa Fluor 568™, Alexa Fluor 594™, Alexa Fluor 610 R-phycoerythrin-streptavidin pH 7.2, Alexa Fluor 647 R-phycoerythrin-streptavidin pH 7.2, Alexa Fluor 633™, Alexa Fluor 647™, Alexa Fluor 660™, Alexa Fluor 680™, Alexa Fluor 700, Alexa Fluor 750, alizarin complexon, alizarin red, allophycocyanine (APC), AMC, AMCA-S, AMCA (aminomethylcoumarin), AMCA-X, aminoactinomycin D, aminocoumarin, aminomethylcoumarin (AMCA), aniline blue, anthrocyl stearate, APC (allophycocyanine), APC-Cy7, APTRA-BTC, APTS, Astrazon Brilliant Red 4G, Astrazon Orange R, Astrazon Red 6B, Astrazon Yellow 7 GLL, atabrin, ATTO-TAG™, CBQCA, ATTO-TAG™ FQ, auramine, aurophosphine, aurophosphine G, BAO 9 (bisaminophenyloxadiazole), BCECF (high pH), BCECF (low pH), BCECF pH 5.5, BCECF pH 9.0, berberine sulfate, beta-lactamase, BFP, BFP/GFP FRET, bimane, bis-acridine orange, bisbenzamide, bisbenzimide (Hoechst), bis-BTC, Blancophor FFG, Blancophor SV, BOBO-3-DNA, BOBO™-1 (2,2′-[1,3-propanediylbis[(dimethylimino)-3,1-propanediyl-[(4H)-pyridinyl-4-ylidenemethylidyn]]bis[3-methyl]-tetraiodide), BOBO™-3 (C₄₅H₅₈I₄N₆S₂), Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy FI, Bodipy FL ATP, BODIPY FL conjugate, BODIPY FL, MeOH, Bodipy FI-ceramide, Bodipy R6G SE, Bodipy TMR, Bodipy TMR-X conjugate, Bodipy TMR-X, SE, Bodipy TR, Bodipy TR ATP, BODIPY TR-X phallacidine pH 7.0, Bodipy TR-X SE, BODIPY TR-X, MeOH, BODIPY TR-X, SE, BOPRO-3, BO-PRO-3-DNA, BO-PRO™-1, BO-PRO™-3, Brilliant Sulphoflavin FF, BTC, BTC-5N, calcein, calcein blue, calcein pH 9.0, calcein/ethidium homodimer, calcium crimson, calcium crimson Ca2+, calcium Crimson™, calcium green, calcium green-1, calcium green-1 Ca2+, calcium green-2, calcium green-5N, calcium green-C18, calcium orange, calcofluor white, carboxy-X-rhodamine (5-ROX), cascade blue, cascade blue BSA pH 7.0, Cascade Blue™ cascade yellow, cascade yellow antibody conjugate pH 8.0, catecholamine, CCF2 (GeneBlazer), CFDA, CFP, CFP (Cyan Fluorescent Protein), CFP/YFP FRET, chlorophyll, chromomycin A, chromomycin A, CI-NERF pH 2.5, CI-NERF pH 2.5, CI-NERF pH 6.0, CI-NERF pH 6.0, CL-NERF, CMFDA, coelenterazine, coelenterazine cp, coelenterazine f, coelenterazine fcp, coelenterazine h, coelenterazine hcp, coelenterazine ip, coelenterazine n, coelenterazine 0, coumarin phalloidin, C-phycocyanine, CPM, CTC, CTC formazan, Cy2™, Cy3.1 8, Cy3.5™, Cy3™, Cy5.1 8, Cy5.5™ Cy5™ Cy7™, cyan 3, cyan 6, cyan GFP, cyclic AMP fluorosensor (FiCRhR), CyQuant, CyQUANT GR-DNA, dabcyl, dansyl, dansylamine, dansylcadaverine, dansylchloride, dansyl DHPE, dansylfluoride, DAPI, dapoxyl, dapoxyl 2, dapoxyl 3, DCFDA, DCFH (dichlorodihydrofluoresceindiacetate), DDAO, DHR (dihydrorhodamine 123), di-4-ANEPPS, di-8 ANEPPS, di-8-ANEPPS (non-ratio), di-8-ANEPPS-lipid, DiA (4-di-16-ASP), dichlorodihydrofluoresceindiacetate (DCFH), DiD, DIDS, dihydrorhodamine 123 (DHR), dihydroethidium, DiI (DiIC18(3)), DiIC₁₈ (“DiD”) dinitrophenol, DiO (DiOC18(3)), DiR, DiR (DiIC18(7)), DM-NERF (high pH), DM-NERF pH 4.0, DM-NERF pH 7.0, DNP, dopamine, DsRed, Draq5, DTAF, DY-630-NHS, DY-635-NHS, EBFP, ECFP, eCFP (Enhanced Cyan Fluorescent Protein), EGFP, eGFP (Enhanced Green Fluorescent Protein), ELF 97, eosine, eosine antibody conjugate pH 8.0, erythrosine, erythrosine ITC, ethidiumbromide, ethidiummonoazide, ethidium homodimer, ethidium homodimer-1 (EthD-1), ethidium homodimer-1-DNA, ethidium homodimer-2, euchrysin, EukoLilght, europium-(III)-chloride, EYFP, eYFP (Enhanced Yellow Fluorescent Protein), Fast Blue, FDA, feulgen (pararosaniline), FIF (Formaldehyde Induced Fluorescence), FITC, FITC antibody, FITC antibody conjugate pH 8.0, flazo orange, fluo-3, fluo-3 Ca2+, fluo-4, fluoroescein, fluorescein (FITC), fluorescein 0.1 M NaOH, fluorescein antibody conjugate pH 8.0, fluorescein dextrane pH 8.0, fluoresceindiacetate, fluorescein pH 9.0, fluoro-emerald, fluoro-gold (hydroxystilbamidine), fluor-ruby, Fluor X, FM 1-43, FM 1-43 lipid, FM 1-43™, FM 4-64, fura red Ca2+, fura red, high Ca, fura red, low Ca, Fura Red™ (high pH), Fura Red™/fluo-3, fura-2, high calcium, fura-2, low calcium, fura-2/BCECF, genacryl brilliant red B, genacryl brilliant yellow 10GF, genacryl pink 3G, genacryl yellow 5GF, GeneBlazer (CCF2), GFP(S65T), GFP red shifted (rsGFP), GFP wild type, non-UV excitation (wtGFP), GFP wild type, UV excitation (wtGFP), GFPuv, gloxalic acid, granular blue, haematoporphyrine, HcRed, hexidium iodide, Hoechst 33258 (bis-benzimide), Hoechst 33258, Hoechst 33342, Hoechst 34580, HPTS, hydroxycoumarin, hydroxystilbamidine, hydroxystilbamidine (FluoroGold), hydroxytryptamine, indo-1, high calcium, indo-1, low calcium, indodicarbocyanine (DiD), indotricarbocyanine (DiR), intrawhite, Cf, JC-1, JO-JO-1 (C₄₇H₅₆I₄N₈O₂), JO-PRO-1, LaserPro, laurodan, LDS 751, LDS 751 (DNA), LDS 751 (RNA), leucophor PAF, leucophor SF, leucophor WS, lissamine rhodamine, lissamine rhodamine B, LIVE/DEAD Kit Animal Cells, LOLO-1 (C₄₇H₅₄Br₂I₄N₈S₂), LO-PRO-1, lucifer yellow, lucifer yellow, CH, lyso tracker blue, lyso tracker blue-white, lyso tracker green, lyso tracker red, lyso tracker yellow, LysoSensor blue, LysoSensor green, LysoSensor green pH 5.0, LysoSensor yellow pH 3.0, LysoSensor yellow/blue, Mag green, Magdala red (phloxin B), Mag-Fura red, Mag-Fura-2, Mag-Fura-5, Mag-Indo-1, magnesium green, magnesium green Mg2+, magnesium orange, malachite green, marina blue, Maxilon Brilliant Flavin 0 GFF, Maxilon Brilliant Flavin 8 GFF, merocyanine, methoxycoumarin, MitoTracker green, MitoTracker green FM, MitoTracker green FM, MeOH, MitoTracker orange, MitoTracker red, MitoTracker red, MeOH, mitramycin, monobromobimane, monobromobimane (mBBr-GSH), monochlorobimane, MPS (Methyl Green Pyronine Stilbene), MRFP, NBD, NBD amine, NBD-X, NBD-X, MeOH, NeuroTrace 500/525, green fluorescent Nissl stain-RNA, Nile blue, EtOH, Nile red, Nile red-lipid, Nissl, nitrobenzoxadidol, noradrenaline, nuclear fast red, nuclear yellow, Nylosan Brilliant lavin EBG, OliGreen®, oregon green, oregon green 488, oregon green 488 antibody conjugate pH 8.0, oregon green 488-X, oregon green 514, oregon green 514 antibody conjugate pH 8.0, Oregon Green™, Oregon Green™ 488, Oregon Green™ 500, Oregon Green™ 514, pacific blue, pacific blue antibody conjugate pH 8.0, pararosaniline (feulgen), PBFI, PE-Cy5, PE-Cy7, PerCP, PerCP-Cy5.5, PE-TexasRed [red 613], phloxin B (Magdala red), phorwite AR, phorwite BKL, phorwite Rev, phorwite RPA, phosphine 3R, PhotoResist, phycoerythrin B [PE], phycoerythrin R [PE], PicoGreen dsDNA quantitation reagent, PKH26 (Sigma), PKH67, PMIA, pontochrome blue black, POPO-1 (2,2′-[1,3-propanediylbis[(dimethylimino)-3,1-propanediyl-1(4H)-pyridinyl-4-ylidenemethyli-dyn]]-bis[3-methyl]-tetraiodide), POPO-1-DNA, POPO-3, POPO™-3 iodide (2,2′-[1,3-propanediylbis[(dimethylimino)-3,1-propanediyl-1(4H)-pyridinyl-4-ylidene-1-propen-1-yl-3-ylidene]]bis[3-methyl]-tetraiodide), PO-PRO-1, PO-PRO-1-DNA, PO-PRO-3, primuline, procion yellow, propidiumiodide (PI), propidiumiodide-DNA, PyMPO, pyrene, pyronine, pyronine B, pyrozal brilliant flavin 7GF, QSY 7, quinacrin mustard, red 613 [PE-TexasRed], resorufin, RH 414, Rhod-2, rhodamine, rhodamine 110, rhodamine 110 pH 7.0, rhodamine 123, rhodamine 123, MeOH, rhodamine 5 GLD, rhodamine 6G, rhodamine B, rhodamine B 200, rhodamine B extra, rhodamine BB, rhodamine BG, rhodamine green, rhodamine phallicidin, rhodamine phalloidin, rhodamine red, rhodamine WT, rhodamine green pH 7.0, rhodol green POPO-1-DNA pH 8.0, ribogreen, rose bengal, R-phycocyanine, R-phycoerythrin (PE), R-phycoerythrin pH 7.5, RsGFP, S65A, S65C, S65L, S65T, sapphire GFP, SBFT, serotonin, Sevron Brilliant Red 2B, Sevron Brilliant Red 4G, Sevron Brilliant Red B, Sevron Orange, Sevron Yellow L, sgBFP™, sgBFP™ (super glow BFP), sgGFP™ (super glow GFP), SITS, SITS (primuline), SITS (stilbene isothiosulphonic acid), SNAFL calcein, SNAFL-1, SNAFL-2, SNARF calcein, SNARF1, sodium green, sodium green Na⁺, spectrum red, SpectrumAqua, SpectrumGreen, SpectrumOrange, SPQ (6-methoxy-N-(3-sulfopropyl)quinolinium), stilbene, sulphorhodamine B can C, sulphorhodamine G Extra, SYBR® Green, SYBR® Green I, SYBR® Green II, SYBR® Gold, SYBR® Safe DNA, SYPRO Ruby, SYTO 11, SYTO 12, SYTO 13, SYTO 13-DNA, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 45-DNA, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Blue-DNA, SYTOX Green, SYTOX Orange, tetracyclin, tetramethylrhodamine (TRITC), Texas Red™, Texas Red-X POPO-1-DNA pH 7.2, Texas Red-X™ conjugate, thiadicarbocyanine (DiSC3), thiazine red R, thiazol orange, thioflavin 5, thioflavin S, thioflavin TCN, thiolyte, tinopol CBS (calcofluor white), TMR, TO-PRO®-1 iodide, TO-PRO®-3 iodide, TO-PRO-1, TO-PRO-1-DNA, TO-PRO-3, TO-PRO-5, TOTO-1 (1-1′-[1,3-propanediylbis[(dimethylimino)-3,1-propanediyl]]bis[4-[(3-methyl-2(3H)-benzothiazolylidene)methyl]]-tetraiodide), TOTO-1-DNA, TOTO-3 (C₅₃H₆₂I₄N₆S₂), TriColor (PE-Cy5), TRITC tetramethylrhodamineisothiocyanate, True Blue, TruRed, ultralite, uranine B, Uvitex SFC, wt GFP, WW 781, X-Rhod-1 Ca2+, X-rhodamine, XRITC, xylene orange, Y66F, Y66H, Y66W, yellow GFP, YFP, YO-PRO®-1 iodide, YO-PRO-1, YO-PRO-1-DNA, YO-PRO-3, YOYO-1 (C₄₉H₅₈I₄N₆O₂), YOYO-1-DNA, YOYO-3 (1,1′-[1,3-propanediylbis[(dimethylimino)-3,1-propanediyl]]bis[4-[3-(3-methyl-2(3H)-benzooxazolylidene)-1-propenyl]]-tetraiodide).

In this context, particularly preferred are 4′,6-diamidino-2-phenylindol, 7-AAD (7-amino-actinomycin D), acridine orange, DNA & RNA, acridine red, Alexa Fluor 594™, Alexa Fluor 610 R-phycoerythrin streptavidin pH 7.2, Alexa Fluor 647 R-phycoerythrin streptavidin pH 7.2, Alexa Fluor 633™, Alexa Fluor 647™, Alexa Fluor 660™, Alexa Fluor 680™, Alexa Fluor 700, Alexa Fluor 750, allophycocyanine (APC), BOBO-3-DNA, BOBO™-3, Bodipy 650/665-X, Cy5.5™, Cy5™, DAPI, DDAO, Draq5, ethidiumbromide, ethidiummonoazide, ethidium homodimer, ethidium homodimer-1 (EthD-1), ethidium homodimer-1-DNA, ethidium homodimer-2, Hoechst 33258, Hoechst 33342, LDS 751, LDS 751 (DNA), LOLO-1, MitoTracker red, Nile blue-EtOH, OliGreen®, PicoGreen dsDNA quantitation reagent, POPO-1-DNA, PO-PRO-1-DNA, propidiumiodide (PI), propidiumiodide-DNA, Ribogreen, SYBR® Green I, SYBR® Green II, SYBR® Gold, SYBR® Safe DNA, SYPRO Ruby, SYTO 60, SYTO 61, SYTO 62, SYTO 63, SYTO 64, SYTOX Orange, Texas Red™ TO-PRO-1-DNA, TO-PRO-3, TO-PRO-5, TOTO-1-DNA, TOTO-3, YO-PRO-1-DNA, YO-PRO-3, YOYO-1, YOYO-1-DNA and YOYO-3.

Most preferred are Pico-Green™ and/or SYTOX Green.

The dose of Pico-Green™ is preferably about 0.01 to 5 μl reagent, preferably 0.05 to 2 μl, more preferably 0.1 to 0.5 μl reagent, most preferably 0.15 to 0.3 μl reagent per measurement with a sample volume of about 40 μl. The amount of reagent used is adjusted to the sample volume. If the detection reagent is provided in solid form, the amount of solid in its content of detection reagent corresponds to the content of dissolved solid in the above-mentioned volume.

Preferably, the opening of the device comprises a one-way valve. Furthermore, the opening preferably comprises a Luer lock.

According to a further preferred embodiment, the device is a disposable device.

The filter is preferably configured so as to separate serum and/or plasma from the blood. Preferably, no lysis of the blood cells, which can influence the measuring result, takes place.

Substances which can remove (bond) bilirubin, hemoglobin or dispersed lipids (also micelles) can be present in the filter. Such substances can be, e.g., titanium dioxides or colestyramine.

When detecting DNA, residual amounts of thrombocytes can be present without having per se a disadvantageous effect on the measurement. When measuring other components, a complete separation of the thrombocytes is advantageous, which can be achieved by using a filter sandwich consisting of a plurality of layers of different filters in a corresponding place of the device.

The device is preferably compatible with commercially available detection devices, in particular in view of its dimensions. The latter devices comprise in particular commercially available photometers, for example the Picofluor, TBS380 (Turner Biosystems, U.S.A.) or the Qubit (Invitrogen, U.S.A.).

Preferably, the device and/or at least partial regions of the device has/have the dimensions of a commercially available cuvette and preferably a diameter of about 10 mm and/or a length of about 50 mm±15 mm or is/are adapted by means of an adapter to the size of a commercially available cuvette. In accordance with the present invention, the term “commercially available cuvette” preferably indicates a cuvette having a diameter of about 10 mm and/or a length of about 50 mm±15 mm.

It is also preferred that the device comprises, in particular for use with the Qubit device, a cuvette in form of a PCR tube (preferably having a useful volume of 0.5 ml).

Moreover, the cuvette can preferably be miniaturized, e.g. consist of a transparent tube of synthetic material or glass and have a filling volume of about 20 to 100 μl, a length of about 5 to 25 mm and an inner diameter of abut 1 to 4 mm.

Preferably, the device comprises at least one venting means. Venting is preferably performed by means of a narrow groove, a channel or a gap, which preferably has a length of only some mm and opens into the measuring chamber or extends therefrom, and whose other end is separated by means of a membrane from the outside air, i.e. environment. This membrane is preferably a semi-permeable membrane that is permeable to air or gas but not to an aqueous fluid. In case a cuvette is used, the semi-permeable membrane is particularly preferably arranged at the lower end of the tube, preferably as a closure of the tube.

The present invention provides a device having an integrated measuring chamber with automatic volumetric dosing. In the filling direction or flow direction of the fluid, the measuring region follows downstream of the filter and, due to its design, arrangement and venting, fills preferably without air bubbles with filtrate or filtered fluid. This occurs preferably in an exactly controllable and correct amount, in agreement with or adjusted to the amount of detection reagent, so that after filling up to the semi-permeable membrane no further flow can take place. The volume is determined by the volumes of the fluid spaces in the cuvette part. The present invention thus allows in particular an automatic volumetric dosing or predetermination of the amount, leading to the advantage that in particular pipetting or other fluid handling is not necessary.

The measuring region can also be partly prefilled, wherein this prefilling can comprise a diluting buffer with a detection reagent. The remaining fillable volume of the measuring region can then be filled with an amount of filtered fluid that is limited by the residual volume and thus defined and, after a short incubation period, can be measured in a fluorescence photometer. After comparing the thus obtained measuring value with a standard, thereby taking into consideration a blank, the amount of a substance contained in the fluid can be determined.

In a further preferred embodiment, the venting means is connected with the measuring chamber by means of a narrow groove or a gap arranged opposite to the inlet opening of the measuring chamber.

Preferably, the measuring region has the shape of or is realized as a conventional (PCR) tube, more preferably comprises a venting means, to which the remaining device is attached.

Preferably, the device comprises at least one second measuring region. Thus, it is possible to simultaneously detect a plurality of measuring values or simultaneously detect one or more calibration values. The device preferably comprises at least a further measuring region for providing a blank or calibration value.

Preferably, the component to be determined interacts with two detection reagents. In this case, the detection or determination of the concentration is performed by means of two detection reagents in a two-step reaction. The detection reagent which bonds first interacts specifically with the component to be determined. This first reagent is preferably coupled to a detectable substance. The detectable substance preferably corresponds to one of the above-mentioned fluorescent dyes.

The second detection reagent is preferably immobilized to a specific region in the measuring region.

The first detection reagent is preferably provided in the device in a region upstream of the measuring region, e.g. in a fluid channel leading thereto, a filter region or a mixing chamber. Before entering the measuring region, the blood or the separated plasma or serum should be brought in contact with the first detection reagent, so that the latter can bond to the substance to be determined. The complex of substance to be detected and first detection reagent then enters the measuring region together with the plasma or serum, where it bonds to a second detection reagent immobilized in a specific region of the measuring region. By accumulation of the complex in this place, the component to be determined can be detected. Excess first detection reagent remains uniformly distributed in the serum or plasma. A control value can be determined, which reflects the concentration of the first detection reagent distributed in the entire solution. This control value can also be programmed by means of a software into a suitable measuring device as threshold or limiting value. In case of this two-step reaction, both reagents used for the detection are referred to as detection reagents, wherein the first detection reagent does not have to change its detectability property when bonding to the component to be determined, as described above. However, it is preferable that the first detection reagent exhibits this property. Both detection reagents directly bond to the component to be determined, so that the actual detection or determination of the component takes place by detecting the first detection reagent, while the second detection reagent fixes the antigen at a position and this position is subjected to a photo-optical measurement after an enrichment caused by a finite perfusion with antigen-containing liquid.

In this two-step reaction, the detection reagents are preferably antibodies or antibody fragments. In case of the first detection reagent, the antibody is coupled to a substance to be detected. For example, R-phycoerythrin (PE), fluoresceinisothiocyanate (FITC), PE-Cy5 allophycocyanine (APC), PE-Texas Red™, peridinin chlorophyll protein (PerCP), PerCP-Cy5.5, APC-Cy7, and/or Texas Red™, etc. are suitable for this purpose.

Components to be detected in a two-step reaction are, e.g., endogenous substances such as interleukin 6 (IL-6), hemoglobin, bilirubin, CRP, lactoferrin, procalcitonin, AT-III, protein C, p24 (HIV) or antibodies. Also exogenous substances such as viruses, bacteria, medicaments, poisons, drugs or fragments of these substances can be determined in this manner.

The device of the invention thus allows the qualitative and/or quantitative determination of a blood component, in particular without further manual or laboratory working steps.

The present invention moreover relates to a method for detecting components in blood, in particular for determining the concentration of components in blood, preferably by using a device of the present invention. The method comprises the steps of (a) providing a device having a measuring region, a filter and a detection reagent, (b) introducing a fluid into the device, and (c) detecting or measuring the concentration of the component by using the device. The step of detecting or measuring the concentration of the component is preferably carried out by using a conventional measuring means, preferably a fluorescence photometer.

The present invention moreover relates to the use of a device and a method of the present invention for determining components in blood such as, e.g., bilirubin, free hemoglobin, IL-6 or p24 (HIV protein), CRP. The use for determining cell-free DNA is also preferred. Hemoglobin or bilirubin is preferably measured by measuring the photo absorption with suitable wavelengths or also by measuring the characteristic autofluorescence of these substances.

The present invention further relates to a kit comprising at least a device according to the present invention and a (fluorescence) standard which can comprise, e.g., DNA. The kit moreover preferably comprises a syringe, preferably with additional components for taking blood from a patient, and/or a measuring means. Furthermore, the kit preferably comprises an operating manual with interpretation aid. The measuring means can moreover comprise a device which is particularly suitable for measuring the samples that have been prepared by means of the device. It can also include a software for storing and processing the data. It can comprise an adapter cable. Moreover, it can comprise a battery pack allowing grid-independent measurements.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows a preferred embodiment of a device of the invention comprising a measuring region, wherein FIG. 1a shows a schematic top view of the device and FIG. 1b a schematic sectional side view of the preferred device;

FIG. 2 shows a schematic top view of a preferred embodiment of the device of the invention with two parallel measuring regions;

FIG. 3 shows a schematic top view of a further preferred embodiment of a device of the invention with two measuring regions;

FIG. 4 shows a schematic top view of a preferred embodiment of a device of the invention with three measuring regions;

FIG. 5 shows schematic top views of further preferred embodiments of the present invention, wherein FIG. 5a shows a preferred embodiment with a flow-optimized measuring region and FIG. 5b shows a preferred embodiment with a different flow-optimized measuring region;

FIG. 6 shows a schematic top view of a preferred embodiment of a device of the invention with integrated immunoassay having a plasma reservoir;

FIG. 7 shows a schematic view of a preferred device of the invention comprising a disk-shaped filter, wherein FIG. 7a shows a schematic top view of the device, FIG. 7b shows a schematic view of the device of FIG. 7a but filled with a fluid and thus with elastically bulged fluid inlet region, and FIG. 7c shows a top view of FIG. 7a;

FIG. 8 shows a schematic top view of a preferred embodiment of a device of the invention;

FIG. 9 shows a schematic top view of a preferred embodiment of a device of the invention;

FIG. 10 shows a schematic top view of a preferred embodiment of a device of the invention;

FIG. 11 shows a schematic top view of a preferred embodiment of a device of the invention;

FIG. 12a shows a schematic side view of a preferred device of the invention, wherein the filter 4 separates the fluid inlet region 5 and the adjoining filtrate region;

FIG. 12b shows a schematic side view of the structure of the device described in FIG. 12a after filling in a sample to be filtered, similar to the device according to FIG. 7b; and

FIG. 12c shows a schematic top view of the structure of the device described in FIGS. 12a and 12b.

### DETAILED DESCRIPTION

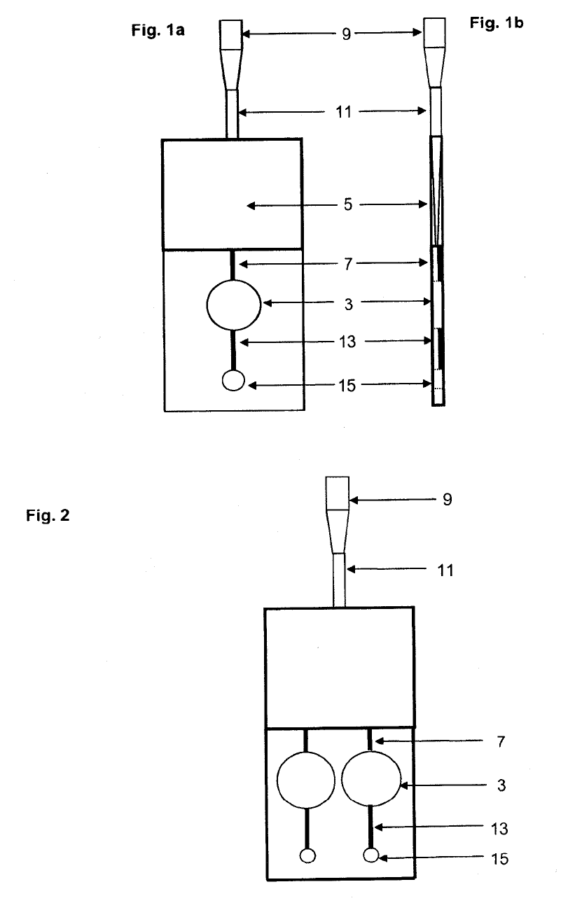

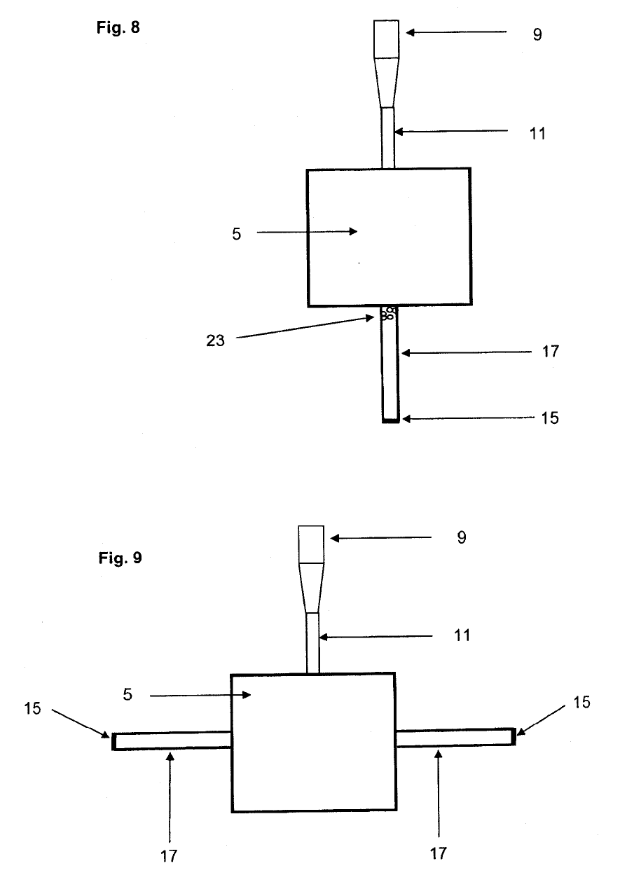

The device according to the invention, as shown, e.g., in FIGS. 1 and 8, serves for detecting, and preferably determining the concentration of, components in fluids. Preferably, the fluid is blood and the component to be determined is DNA.

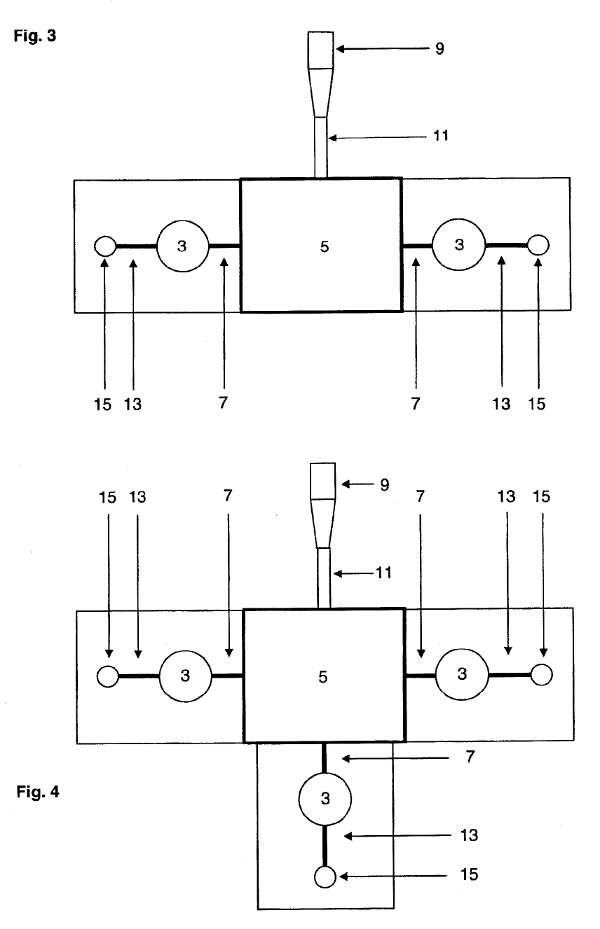

The device according to FIGS. 1 to 7 comprises a measuring region 3 as well as a filter region 5 being in fluid communication therewith. The filter region 5 and the measuring region 3 are preferably connected with each other via a first fluid channel 7. The device 1 preferably also comprising an opening 9 realized preferably as a Luer lock and more preferably comprises a one-way valve. The opening 9 is located preferably directly at the fluid inlet region of the filter region 5 or is connected therewith via a hose or tube 11, preferably comprising a polymer, preferably of polyvinylchloride (PVC) or polyethylene (PE).

The filter region 5 is preferably elastic and bag-shaped and moreover or additionally preferably made of a soft PVC, PE, a copolymer or also composite polymer. The opening 9 is preferably realized such, for example by forming a Luer lock, that a commercially available syringe (not shown) can be connected thereto. For example, fluid filled-in by means of such a syringe, in particular blood, is introduced through the opening 9 and optionally the tube 11 into the filter region 5. The filter region 5 is preferably realized such that when a predetermined amount or predetermined volume of fluid is introduced, it expands in a predetermined manner which, in turn, causes a predetermined pressure to be exerted on the interior of the filter region 5. Materials, volume to be inserted and required volume and the like are preferably adjusted accordingly. Providing the opening 9 with a one-way valve prevents the pressurized fluid in the fluid inlet region or the filter region 5 from escaping.

Caused by the pressure now prevailing in the filter region 5, the fluid contained therein is pressed through a filter, preferably a special membrane which is preferably welded into the filter region 5.

In addition to the opening 9, the filter region 5 preferably comprises an outlet opening into the fluid channel 7, which in turn leads to the measuring region 3. The filter is preferably arranged such that the pressurized fluid in the filter region 5 is transported through the filter (not shown) into the fluid channel 7 and thus into the measuring region 3. The thus filtered fluid, preferably the blood plasma and particularly preferably a predetermined volume of blood plasma is thus provided in the measuring chamber 3.

The above statements analogously also apply to the preferred embodiments according to FIGS. 8 to 11, except for the fact that the parts measuring region 3, fluid channel 7, venting channel 13, as described in connection with FIGS. 1 to 6, are integrated in one part 17 which is preferably closed by means of a semi-permeable membrane 15.

The device further comprises a detection reagent, preferably Pico-Green™, which is provided in the measuring chamber 3 and/or in the fluid channel 7 in such a manner that it comes in contact and in particular interacts with the blood plasma or serum flowing through the fluid channel and/or with the blood plasma or serum collected in the measuring chamber 3, preferably such that it is possible to detect, in particular to determine the concentration of, components in the fluid or blood plasma.

For this purpose, the device and in particular the volumes of measuring region 3, filter region 5 and fluid channel 7 (or part 17) as well as the properties of the filter region 5 in view of elasticity and pressure generation as well as of the filter in view of filtering and flow properties are adjusted such that a predetermined amount of blood plasma, which interacts with a predetermined amount of detection reagent, is provided in the measuring chamber 3 and/or in the fluid channel 7 (or part 17).

Preferably, the inner region of the measuring chamber 3 and/or the fluid channel 7 (or part 17), which comes in contact with the blood plasma, is at least partly coated with a detection reagent, which preferably comprises or consists of Pico-Green™ or SytoxGreen. Moreover, also a quickly soluble pellet comprising a detection reagent can be positioned in the region of the measuring chamber 3 or the fluid channel 7 (or part 17).

Preferably, the measuring region 3 opens into a channel, preferably a venting channel 13. Such a channel preferably opens into a venting opening or venting recess 15, which is preferably arranged in the outer region of the device and sealed towards the environment by means of a semi-permeable membrane (not shown). Such a membrane is preferably characterized in that from the inner side of the device it is permeable to air or gaseous media but not to liquid media or blood plasma. Accordingly, the channel and the venting region 15 are adapted to transport excess air in the device 1 to the outside into the environment when the filter region 5 and the fluid channel 7 and the measuring region 3 are filled, in other words adapted to vent the device 1. Thus, it is advantageously possible to introduce in particular a defined liquid volume into the measuring region.

By means of the device of the invention, it is thus possible to automatically fill the measuring region 3 with a defined amount of blood plasma and mix it with a detection reagent. The device and the fluid, in particular blood plasma, in the measuring region can be detected in particular photo-optically, in accordance with the above description, e.g., for detecting free DNA by means of an intercalating fluorescent dye such as, e.g., Pico-Green™

The detection reagent is preferably applied to the inner side of the measuring region 3, e.g. a measuring chamber, or the measuring channel, preferably by means of an ink jet or a technique equivalent to an ink jet printer, and dried. Alternatively or additionally, the detection reagent is provided in the measuring chamber as dust or pellet, or, e.g., in the supply channel as a readily soluble material. The device preferably has a diameter of about 10 mm and a length or height of about 50 mm±15 mm.

The measuring region is preferably at least partly transparent or translucent, in particular for guaranteeing an optical detection of the component to be detected or determined, preferably by means of a fluorescence measurement.

The above description, which particularly relates to FIG. 1, analogously also applies to the preferred embodiments according to FIG. 2 to 6 (or 7). The preferred embodiments according to FIGS. 2 to 4 show devices which basically correspond in view of structure and mode of operation to the device described in connection with FIG. 1, which however comprise several measuring regions 3, preferably 2 or 3 measuring regions, wherein these measuring regions can be arranged in different manners. In accordance with the general structure and the mode of operation, reference is made to the above description of FIG. 1. In connection with the design comprising several measuring regions 3, it is only pointed out that the different measuring regions, or the fluid channels 7 extending from the common filter region 5, and the measuring regions 3, venting channels 13 and venting regions 15 following in the flow direction are suitable for carrying out a plurality of parallel measurements or detections or determinations, wherein any of the measuring regions 3 and/or fluid channels 7 can comprise the same or different detection reagent(s).

The above description, which particularly relates to FIG. 1, analogously also applies to the preferred embodiments according to FIGS. 8 to 10. The preferred embodiments according to FIGS. 8 to 10 show devices which basically correspond in view of structure and mode of operation to the device described in connection with FIGS. 1 to 7.

Preferably, at least one of the measuring regions, also referred to as blank measuring region, is provided for determining a blank. A blank is a value used for comparing the sample to be measured. For example, filtered blood plasma can be measured in the blank measuring region without reagent as blank, which then indicates, e.g., an autofluorescence. A calibration value can be formed, preferably by measuring a fluid of the same kind to which standard amounts of the substance to be measured are added. More preferably, a calibration value can be formed by measuring a cuvette which is filled with/consists of a liquid that is optically similar to the blood plasma or also a synthetic material and which exhibits a stable defined fluorescence. Based on these values, the corresponding measuring value of the sample(s) is evaluated. For example, a standard measurement can be carried out daily, and always a blank and a measuring value of the analyte of interest can be obtained from the patient.

By means of the corresponding preferred embodiment it is thus possible to simultaneously carry out parallel identical measurements or parallel different measurements. As already described in connection with the preferred device of measuring FIG. 1, also in the preferred embodiments shown in FIGS. 2 to 6 (or 7) the volumes of measuring region 3, filter region 5 and fluid channels 7 as well as the properties of the filter region 5 in view of elasticity and pressure generation as well as of the filter in view of the filtering and flow properties are adjusted such that a predetermined amount of blood plasma, which interacts with a predetermined amount of detection reagent(s), is present in the measuring chambers 3 or in the fluid channels 7.

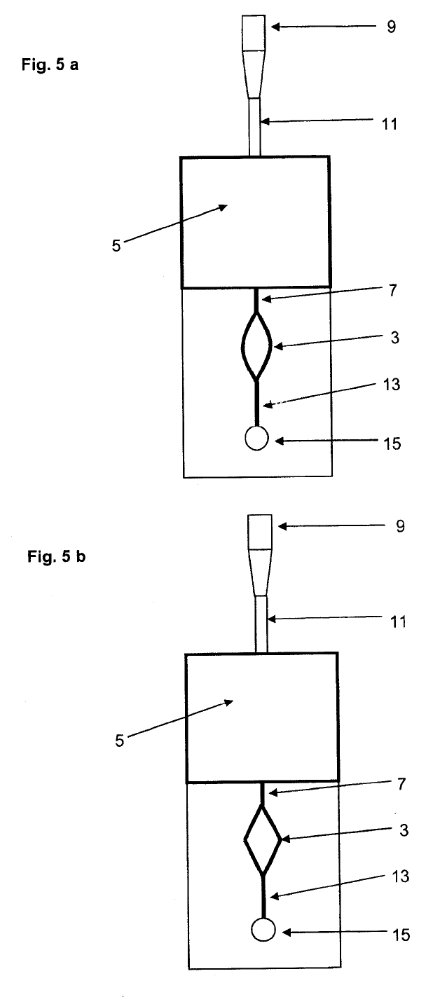

FIG. 5 in turn shows a preferred embodiment of a device of the invention comprising a measuring chamber, wherein only exemplarily a changed, flow-optimized measuring region 3 (see FIG. 5a, FIG. 5) is shown. This is to clarify that the geometry of the measuring region 3 is not considered as being restrictive but can have any desirable different shape as long as the described mode of operation is guaranteed.

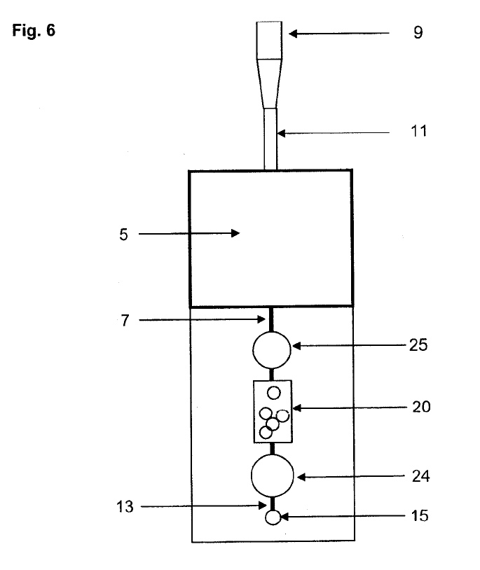

Also FIG. 6 shows a further preferred embodiment of a device of the invention according to which a further chamber 20 is arranged downstream of a mixing chamber 25 which preferably comprises a reagent which bonds the component(s) to be measured. In this chamber 20, a detection reagent, e.g. an antibody, is firmly bound to a substrate comprising preferably at least partly silicon dioxide (e.g. glass) or polystyrene or polyurethane. A component to be measured, e.g. an antigen, then bonds in the filter region 5, mixing chamber 25 and/or the channel 7 to a first detection reagent, e.g. an antibody marking fluorescence, and when further flowing through the chamber 20 is fixed to the substrate by a further antibody bound to the substrate, and thus the presence of the antigen and/or its concentration is determined by a fluorescence-photometric measurement of the chamber 20. Both detection reagents can bond to different regions of the component to be determined, in case of antibodies to different epitopes of the antigen.

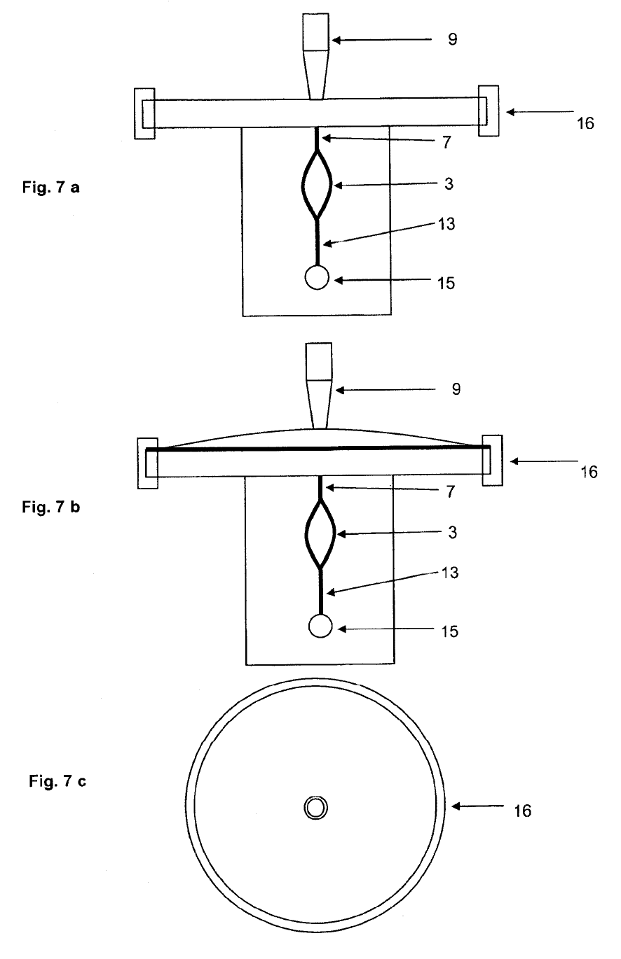

FIG. 7 shows a schematic view of a preferred device of the invention comprising a disk-shaped filter 16. FIG. 7a shows a schematic top view of the device and FIG. 7b shows a schematic view of the device of FIG. 7a but filled with a fluid and thus with elastically bulged fluid inlet region. FIG. 7c shows a schematic view of the device of FIG. 7a, as viewed from the top of FIG. 7a. The remaining arrangement as shown corresponds to the arrangements already described.

FIG. 8 shows a schematic top view of a preferred embodiment of a device of the invention comprising a tube-shaped cuvette 17, which can contain, e.g., a pelleted, readily soluble reagent 23. Also by means of this device of the invention it is possible to fill the measuring region with a defined amount of blood plasma and to mix it with a detection reagent. Here, the combination of the described parts measuring region 3, fluid channel 7 and venting channel 13 according to the embodiments shown in FIGS. 1 to 6 is integrated or arranged in the part 17. The part 17 is preferably provided with a closure formed by a semi-permeable membrane 15. The device and fluid in the measuring region, preferably blood plasma, can in particular be detected photo-optically. The tubular or hose-shaped, transparent measuring cuvette has a length of about 20 mm, an inner diameter of about 1 to 4 mm and an outer diameter of about 2 to 6 mm.

FIG. 9 shows a schematic top view of a preferred embodiment of a device of the invention comprising two tubular cuvettes 17, wherein one of said cuvettes can be provided with a detection reagent, while by means of the other one comprising no reagent a blank can be measured.

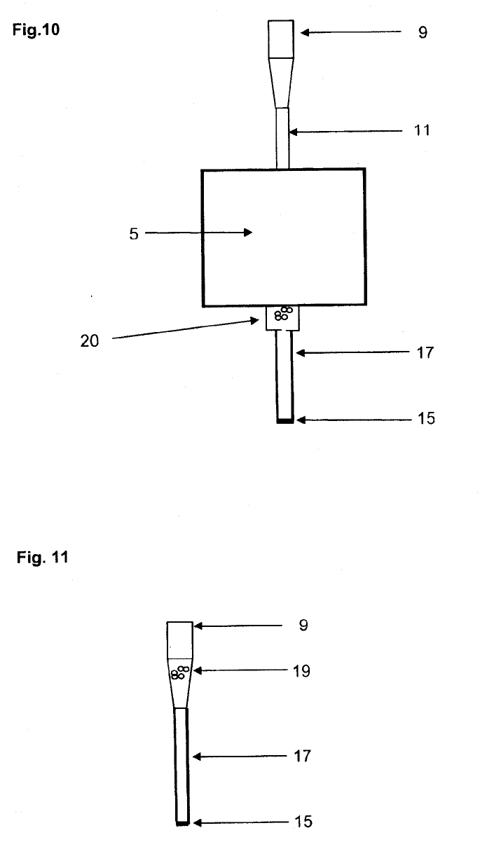

FIG. 10 shows a schematic top view of a preferred embodiment of a device of the invention comprising a mixing chamber 20 which can contain a reagent, wherein the reagent in the chamber 20 mixes in a particularly preferred manner with the filtrate.

FIG. 11 shows a schematic top view of a preferred embodiment of a device of the invention by means of which the measuring region can be automatically filled with a defined amount of blood plasma produced previously by means of common methods. The tubular or hose-shaped measuring cuvette has a length of about 20 mm and an outer diameter of about 2 to 6 mm and can comprise a reagent in the region 19.

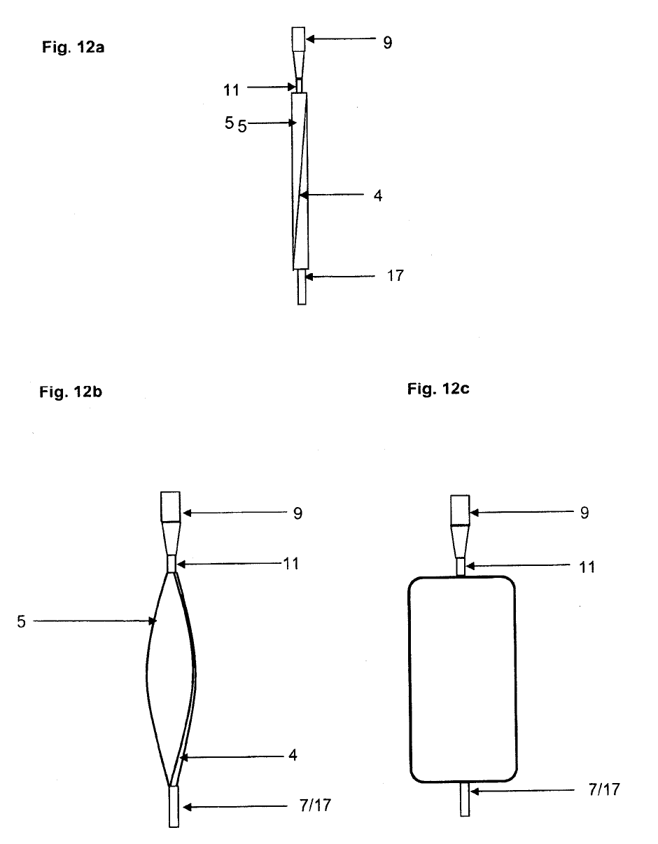

FIG. 12a shows a schematic side view of a preferred device of the invention in which the filter 4 separates the fluid inlet region 5 and the filtrate region passing into a part 7 or 17. The filter is preferably continuously connected with the walls or films limiting the fluid inlet region as well as the measuring region and/or the filtrate region, preferably by gluing or welding.

Part 7 or 17 of the described embodiments comprises, e.g., a capillary for venting and/or measuring and/or the measuring region.

FIG. 12b shows a schematic view after filling the device of FIG. 12a. FIG. 12c shows a view of the wide side of FIGS. 12a and 12b. The devices have elastic walls which are preferably formed of films or comprise one or more film regions and which limit the fluid inlet region 5 as well as the filtrate region. The filter 4 can either stick closely to the walls of the device or be folded. In both states the possibly fragile filter membrane is supported mechanically. By filling the fluid inlet region through the opening 9 and, in particularly preferred embodiments by the presence of a valve upstream of the fluid inlet region, there is a pressure difference between the fluid inlet region and the filtrate region over the filter. If the region downstream of the filter, e.g. the filtrate region and/or measuring region, is also formed by an elastic film, this preferred embodiment of the invention advantageously leads to a particularly small dead space volume and, as a consequence, a very small amount of filtrate is required for carrying out the measurement. The device moreover comprises, e.g., a capillary or opening 17 suitable for transmitting, measuring and/or removing the filtrate.

During operation, the device is preferably filled through the opening 9 or the fluid inlet region. A syringe is preferably used for this purpose. According to a preferred embodiment, the opening 9 comprises a one-way valve, for example a Luer lock. By applying pressure on the sample, which pressure is applied in the described embodiment preferably manually by means of the syringe, and by the thus introduced sample, the volume in the fluid inlet region increases. In other words, by the material introduced under pressure, the wall or film, which at least partially forms the fluid inlet region, is expanded. Thus, on the one hand, a sufficient space for the sample volume to be introduced is formed. On the other hand, the applied filling pressure results in an expansion of the film or the elastic material so that the fluid inlet region is filled with a pressurized sample material. The sample material pressurized by the expanded film or the expanded elastic material is thus pressed through the filter and filtered. The filtrate exiting the filter on the other side thus passes the filter and reaches the filtrate region and/or measuring region. This region is also preferably at least partially formed by an elastic material, e.g. an elastic film, so that its volume is formed in accordance with the entering filtrate. This leads to the advantages described above.

Thus, the present invention provides an advantageous device, method and kit, which overcome the disadvantages of the prior art. In particular, by means of the present invention it is possible to separate plasma or serum from whole blood and to analyze substances contained in the serum or plasma without having to carry out centrifugal steps or similar laboratory processing steps with the gained serum or plasma. Moreover, the present invention provides a device, method and kit which can be handled or carried out easily, safely and reliably, can be used or carried out in particular by laymen or staff that has not been medically trained, and can be produced, carried out and/or stored easily and in a cost-efficient manner.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

1. A device for detecting components of blood, in for particular determining the concentration of components in blood, the device comprising:
a measuring region;
a filter and/or filter region;
at least one detection reagent for interaction with the components;
an opening for introducing a fluid, the filter being arranged between the opening and the measuring region; and
a fluid inlet region,
wherein the fluid inlet region and/or the filter region is/are formed or limited at least partially by an elastic region, which comprises a film.

2. The device according to claim 1, wherein the fluid inlet region is arranged between the opening and the measuring region, preferably between opening and filter.

3. The device according to claim 1, wherein the component to be detected or determined is a substance occurring in organisms.

4. The device according to claim 1, wherein the component to be detected or determined is a biological molecule selected from the group comprising DNA, RNA, proteins, hormones, cytokines.

5. The device according to claim 1, wherein the component to be detected or determined is a medicament or drug.

6. The device according to claim 1, wherein the presence and/or concentration of the component in the measuring region can be determined by via luminescence, fluorescence, autofluorescence, chemiluminescence, electrochemiluminescene, spectral absorption photometry and/or bioluminescence.

7. The device according to claim 1, wherein the filter is adapted to separate solid components of the blood flowing through the filter and to separate the solid and liquid phases of the blood from each other.

8. The device according to claim 1, wherein the fluid inlet region is adapted to exert a pressure lying above ambient pressure on the blood.

9. The device according to claim 1, wherein the device is adapted to introduce the blood under pressure through the filter into the measuring region.

10. The device according to claim 1, wherein a pressure lying below ambient pressure is prevailing in the device.

11. The device according to claim 1, wherein the detection reagent is provided in the measuring region.

12. The device according to claim 1, wherein the detection reagent interacts directly or indirectly with the component.

13. The device according to claim 1, wherein the detection reagent changes its optical properties when interacting with the component to be determined.

14. The device according to claim 1, wherein the detection reagent is selected from the group comprising Pico-Green™, Alexa dyes, ethidium bromide and SYBR® or Sytox® dyes.

15. The device according to claim 1, wherein the opening comprises a one-way valve.

16. The device according to claim 1, wherein the opening comprises a Luer lock.

17. The device according to claim 1, wherein the device is a disposable device.

18. The device according to claim 1, wherein the filter is adapted to separate serum or plasma from the blood.

19. The device according to claim 1, wherein the device is compatible with commercially available detection devices.

20. The device according to claim 1, wherein the device has the dimensions of a commercially available cuvette or another commercially available measuring vessel and has a diameter of about 10 mm and/or a length of about 50 mm±15 mm or is adapted by an adapter to the size of a commercially available cuvette.

21. The device according to claim 1, wherein the device comprises at least one vent.

22. The device according to claim 21, wherein the vent is connectable with the measuring chamber by a narrow groove or a gap.

23. The device according to claim 1, wherein the measuring region of the device is in the form of a tube comprising a vent.

24. The device according to claim 1, wherein the device comprises at least a second measuring region.

25. The device according to claim 1, wherein the device comprises at least one region for providing a blank or calibration value.

26. The device according to claim 1, wherein the component to be determined interacts with two detection reagents.

27. The device according to claim 26, wherein the first detection reagent is provided in the device upstream of the measuring region.

28. The device according to claim 26, wherein the second detection reagent is immobilized to a specific region in the measuring region.

29. A method for detecting components in blood, in particular for determining the concentration of components in blood by using a device according to claim 1, the method comprising:
providing a device having the measuring region and the detection reagent;
introducing blood into the device; and
detecting or measuring the concentration of the component via the device.

30. The method according to claim 29, wherein the blood is introduced by via a syringe, wherein a pressure required for filtering is applied.

31. Use of a device and/or a method according to claim 1 for determining components in blood.

32. A kit comprising the device according to claim 1 and a fluorescence standard.

33. The device according to claim 1, wherein the component to be detected or determined is a protein.
